(19) United States
(12) Patent Application Publication
(10) Pub. No.: US 2007/0021734 A1
(43) Pub. Date: Jan. 25, 2007

(54) BIOELECTRO-OSMOTIC ENGINE FLUID DELIVERY DEVICE

(76) Inventor: SAI BHAVARAJU, Salt Lake City, UT (US)

Correspondence Address: CERAMATEC, INC., 2425 SOUTH 900 WEST, SALT LAKE CITY, UT, 84119, US

(21) Appl. No.: 11/457,010

(22) Filed: Jul. 12, 2006

Related U.S. Application Data
(60) Provisional application No. 60/700,022, filed on Jul. 15, 2005.

(51) Int. Cl. A61K 9/22

(52) U.S. Cl. 604/891.1

(57) ABSTRACT

Disclosed are embodiments of a fluid delivery device that may be used in implantable applications. In one illustrative embodiment, the fluid delivery device may include an electro-osmotic pump having a biocompatible electrode configured to oxidize body fluid.

### RELATED APPLICATIONS

This application claims the benefit under 35 U.S.C § 119(e) of U.S. Provisional Patent Application No. 60/700,022 filed Jul. 15, 2005, and titled “Bioelectro-Osmotic Engine Fluid Delivery Device,” which is incorporated herein by reference.

### BRIEF DESCRIPTION OF THE DRAWINGS

Understanding that drawings depict only certain embodiments of the disclosure and are therefore not to be considered limiting of its scope, the embodiments will be described and explained with additional specificity and detail through the use of the accompanying drawings in which:

FIG. 1 is a block diagram of one embodiment of a cationic electrokinetic-based fluid delivery device including an electro-osmotic engine having a biocompatible electrode.

FIG. 2 is a block diagram of one embodiment of an anionic electrokinetic-based fluid delivery device including an electro-osmotic engine having a biocompatible electrode.

### DETAILED DESCRIPTION

In the following description, numerous specific details are provided for a thorough understanding of specific embodiments. However, those skilled in the art will recognize that embodiments can be practiced without one or more of the specific details, or with other methods, components, materials, etc. In some cases, well-known structures, materials, or operations are not shown or described in detail. Furthermore, the described features, structures or characteristics may be combined in any suitable manner in a variety of alternative embodiments.

Disclosed are embodiments of systems, methods, and apparatus relating to fluid delivery devices. The term “fluid” is meant to include a liquid, gel, paste, or other semi-solid state or flowable material that is capable of being delivered out of a reservoir. In some embodiments, these fluid delivery devices are capable of delivering a small amount of a beneficial agent over a period of time. The term “beneficial agent” is meant to include, but is not limited to, any therapeutic agent or drug, medicament, vitamin, lubricant, chemical agent or solution that can be administered to produce a desired, usually beneficial effect.

In some embodiments, the fluid delivery devices may be implantable in an animal. In other embodiments, the fluid delivery devices may be disposed outside of the body of an animal, while remaining in fluid communication with the body surface or internal to the body of an animal, such as through a needle, catheter and the like. The term “animal” is meant to include organisms of the kingdom Animalia including, but not limited to, mammals (e.g., humans), birds, fish, etc.

In some embodiments, the fluid delivery device is configured to prevent exposure of metal ions to the interior of the human body when the device is used in implantable applications. This may be useful from, for example, toxicology, tissue response, encapsulation, and protein-interaction perspectives in some specific implant applications. In some embodiments, this may be accomplished through the use of biocompatible components. The term “biocompatible” means that a component or system does not cause significant injury, toxic or immunologic reaction to living tissue.

Exemplary fluid delivery devices having components that may be used in connection with embodiments of the systems, devices, and methods disclosed herein can be found in U.S. Patent Application Publication No. 2003/0205582 titled “Fluid Delivery Device Having an Electrochemical Pump with an Anionic Exchange Membrane and Associated Method,” U.S. Pat. No. 5,744,014 titled “Storage Stable Electrolytic Gas Generator for Fluid Dispensing Applications,” and U.S. Pat. No. 5,707,499 titled “Storage-stable, Fluid Dispensing Device Using a Hydrogen Gas Generator.” Each of the foregoing references are hereby incorporated by reference.

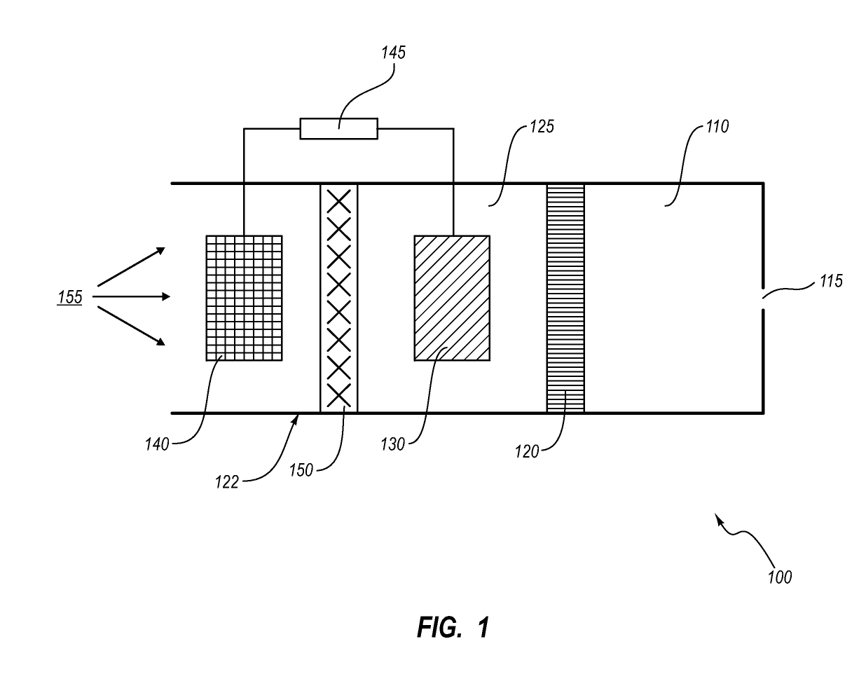

Further details of specific illustrative embodiments will now be described with reference to the accompanying drawings. FIG. 1 depicts an embodiment of a fluid delivery device 100. Fluid delivery device 100 comprises a fluid reservoir 110. The fluid reservoir 110 may comprise a chamber having fixed, rigid or semi-rigid walls, or alternatively may comprise a bag or bellows or the like.

The fluid reservoir 110 may house a beneficial agent such as a drug. Fluid reservoir 110 includes a port 115 or orifice, through which the fluid stored in fluid reservoir 110 may be dispensed. It should be understood that, in some embodiments, port 115 may be in fluid communication with a catheter, tube, or other fluid delivery component. A piston 120 or other displaceable member may be positioned to slide within or otherwise apply pressure to reservoir 110 so as to be capable of driving the fluid stored in reservoir 110 through port 115. Alternative displaceable members include, but are not limited to, a bellows, a bladder, a bag, a diaphragm, a plunger, and combinations thereof.

Fluid delivery device 100 also includes an electrochemical device, such as an electrochemical engine or pump 122, which is configured to provide a force against the piston 120 or other displaceable member to facilitate dispensing fluid out of the fluid reservoir port 115. In one embodiment, such as the embodiment of FIG. 1, the electrochemical pump 122 is an electro-osmotic pump capable of transporting water. An electro-osmotic pump may move fluid by the application of an electric field through an electro-osmotic mechanism.

The electrochemical pump 122 in the embodiment of FIG. 1 is a cationic electrokinetic (“CATEK”) system. However, as will be described further, it should also be understood that the principles set forth herein are applicable to anionic electrokinetic (“ANEK”) as well as CATEK systems.

The electrochemical pump 122 includes a first electrode 130 which may comprise a cathode, and a second electrode 140 which may comprise an anode. Electrodes 130 and 140 may be connected via circuit element 145. Circuit element 145 may comprise a resistor or series of resistors. In some embodiments, the resistor(s) may be replaceable or adjustable so as to vary the rate at which the electrochemical device operates. For example, an adjustable resistor may control the fluid delivery rate. In other embodiments, the circuit element 145 may comprise a switch or other electrical component including a component which merely completes the circuit between electrodes 130 and 140.

An ion exchange membrane 150 is positioned between the two electrodes 130, 140 to provide ionic communication therebetween. In the embodiment of FIG. 1, the ion exchange membrane 150 comprises a cation exchange membrane. The cation exchange membrane 150 allows the transport of cations from adjacent the anode 140 to a driving chamber 125 housing the cathode 130. In the embodiment of FIG. 1, the anode 140 is disposed outside of the driving chamber 125, and may be exposed to body fluid 155 and/or a saline solution.

Once the electrochemical pump 122 is activated, sodium ions present in body fluid 155 and/or saline solution migrate under the influence of the electric field through the cation exchange membrane 150 (e.g., those sold under the Nafion® brand) towards the cathode 130 in the driving chamber 125. During passage of the sodium ions through the cation exchange membrane 150, a sheath of water molecules is entrained with the sodium ions such that, at the opposite side of the membrane 150, an additional amount of water is generated. This electrokinetic water transport is known in the art as electro-osmotic transport. The water molecules transported into the driving chamber 125 generate pressure which an be used to drive piston 120 (or other displaceable member) and deliver the fluid within reservoir 110.

The steady buildup of ions in the driving chamber 125 due to the transport of sodium ions and the anions produced at the cathode 130 induces further water transport through an osmotic effect. For instance, if a metal chloride cathode were used as the cathode 130, an equilibrium concentration of sodium chloride may be established in the driving chamber 125 after a period of operation resulting in water transport via the osmotic effect. The cation exchange membrane 150 may allow some back diffusion of sodium chloride from the driving chamber 125 toward the anode 140. Thus, a steady-state flux of water transport into the driving chamber 125 is established by combined electro-osmotic and osmotic effects.

In a traditional CATEK system, the anode may comprise zinc or other electropositive metal or metal containing electrode. When oxidation occurs at the anode of conventional systems, zinc is dissolved according to the equation:
Zn→Zn²⁺+2e⁻ (1)

However, in an implantable device, the zinc electrode may be exposed to body fluid 155. Consequently, oxidation products of zinc, such as zinc chloride, zinc carbonate and zinc oxide may migrate into the surrounding body fluid. The presence of zinc or certain other metal-containing species may create a toxicological response from the surrounding tissue. Furthermore, the presence of zinc might influence encapsulation behavior and facilitate unwanted protein interaction. Consequently, use of an anode 140 that is biocompatible may be desired.

According to the present disclosure, a biocompatible electrode, such as a biocompatible anode 140 may be used to oxidize a fuel present in the body of an animal, such as a human. In some embodiments, the anode is not disposed within a separate chamber of the electrochemical pump 122 volume and is exposed to body fluid 155. Furthermore, the consumable zinc anode may be replaced by a small current collector on which, for example, the glucose oxidizing electrocatalyst is placed. Therefore, the ratio of the electro-osmotic engine volume to volume of fluid to be dispensed may be reduced compared to conventional devices. However, it is understood that the anode 140 may be disposed in a separate chamber that is exposed to body fluid through a membrane, or is impermeable and houses some form of anolyte or other acceptable solution.

One exemplary embodiment of an anode using fuel present in the body includes the use of a glucose anode 140 that oxidizes glucose present in body fluid 155. The glucose anode 140 may be an enzymatic anode utilizing an enzyme based electrocatalyst. Alternatively, other fuels present in the body, such as lactate, may be used instead of, or in combination with, glucose. Furthermore, traditional metal, polymer, carbon and ceramic based electrocatalyst may be used instead of enzymatic electrodes. According to one embodiment, where glucose is used as the fuel, the glucose oxidation reaction is accomplished according to the equation:
Glucose→Gluconolactone+2H⁺+2e⁻ (2)

Additionally, glucose may be oxidized to produce CO₂, protons and electrons. By way of example, the enzyme based electrocatalyst for oxidizing glucose may comprise the electrostatic adduct of glucose oxidase (Gox), a polyanion at physiological pH, and the polycationic redox polymer poly-(N-vinyl imidazole), partially quarternized with 2-bromoethylamine and partially complexed with [(Os(da-bpy)₂Cl]^{+/2+}(where da-bpy=4,4′-diamino-2,2′-bipyridine).

In some embodiments, an enzymatic anode may comprise a conductive support, such as a metal or carbon, on which the enzyme is immobilized. The enzyme can either directly oxidize the body fuel, such as glucose, using direct electron transfer as the electron transfer mechanism, or the enzyme may utilize a redox mediator via mediator electron transfer as the electron transfer mechanism. As will be discussed further, enzymatic electrodes may be used as cathodes as will as anodes.

In those embodiments utilizing direct electron transfer, no mediator is required since the enzyme can directly interact with a substrate forming a molecular transducer that converts a chemical signal directly into an electrical signal. Exemplary direct electron transfer enzymes include, but are not limited to laccase, lactate dehydrogenase, peroxidase and hydrogenase. Laccase-based enzyme anodes may be capable of oxidizing both glucose and lactate. However, lactate dehydrogenase-based enzyme anodes may be selective for lactate and not glucose.

Exemplary mediator electron transfer enzymes include, but are not limited to, glucose oxidase and bilirubin oxidase. Mediators may include AND(P)⁺ for anodic mediators, ABTS (2,2′-azinobis-(3-ethylbenzothiazoline-6-sulfonate)) for cathodic mediators, and labile osmium complexes for both anodic and cathodic mediators.

According to one embodiment the cathode 130 is a metal chloride electrode, such as silver chloride. At electrode 130, silver chloride may be reduced to metallic silver, thereby releasing chloride ions into the solution around electrode 130 according to the equation:
2AgCl+2e⁻→2Ag+2Cl⁻ (3)

Alternatively, other metal chlorides may be used as the cathode 130. For example, high oxidation state cupric, ruthenium, platinum, palladium, iridium or gold chlorides, among others, may be used. Furthermore, reducible cathodes such as MnO₂ or AgO may also be used.

According to another embodiment, the cathode 130 is an oxygen-reducing cathode. Soluble oxygen present in body fluid 155 will diffuse through the cation ion-exchange membrane and is reduced at the cathode 130. In one example, the oxygen-reducing cathode 130 is an enzymatic cathode. Examples of oxygen-reducing enzymatic cathodes include, but are not limited to, bilirubin oxidase, laccase, and cytochrome c oxidase. Furthermore, traditional fuel cell cathodes, such as silver, platinum or metal oxide loaded on a conductive carbon oxygen reducing cathodes may also be used. Much like the anode described herein, the cathode may also be biocompatible.

The use of enzymatic electrodes, as either the anode 140, cathode 130 or both, provides the benefit of having a reduced ratio of the electro-osmotic engine volume to volume of fluid to be dispensed. Enzyme base electrocatalysts typically require a smaller surface area then traditional metal electrodes. For examples, in one embodiment, an oxygen reducing exzymatic cathode using an osmium complex mediator may be wired using a rapidly electron diffusing polymer. The wired structure may allow oxygen reduction by biological oxygen demand by 100-fold compared to platinum cathodes. Hence, a smaller amount of catalyst is needed.

Furthermore, advances in the technology of enzymatic electrodes now provides that a separate power source is not necessarily required. Enzymatic electrodes are now a practical option whereas in previous years they were not as viable as an alternative.

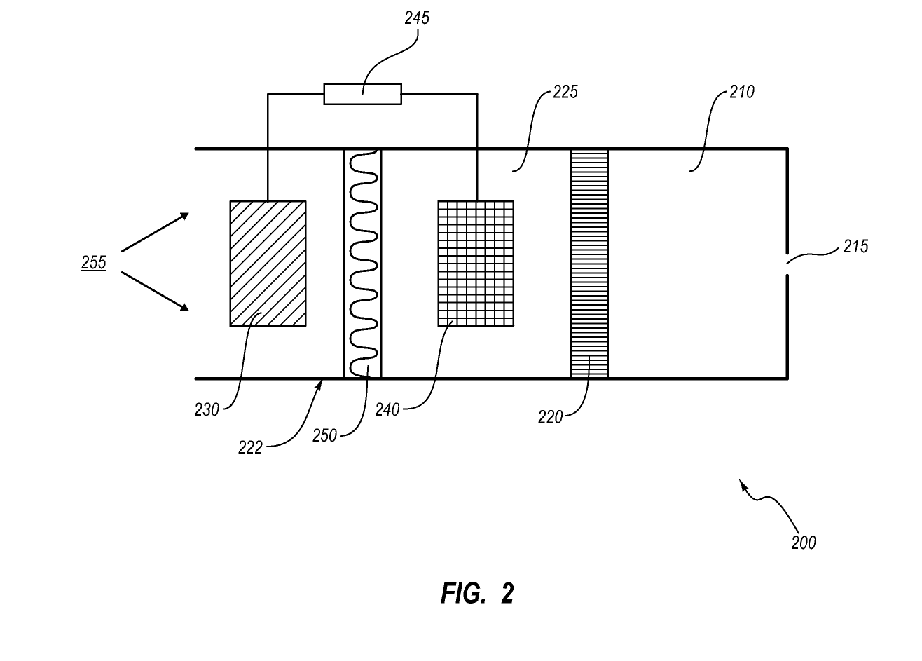

FIG. 2 depicts another embodiment of a fluid delivery device 100. Like fluid delivery device 100, fluid delivery device 200 includes a fluid reservoir 210 with a port 215 and a displaceable member such as a piston 220 to facilitate dispensing fluid out of fluid reservoir 210. The fluid delivery device 200 also includes an electrochemical pump 222 which, in one embodiment, may be an electro-osmotic pump comprising a first electrode 230 coupled to a second electrode 240 via circuit 245. However, in the embodiment of FIG. 2, an anion exchange membrane 250 may be positioned between electrodes 230 and 240. Electrode 230 may be a cathode that is located outside of driving chamber 225. Electrode 240 may be an anode that is disposed inside driving chamber 225, or otherwise separated from direct exposure to body fluid 255 via the anion exchange membrane 250. The fluid delivery device 200 is, therefore, an ANEK system.

In an ANEK system, once the electrochemical pump 222 is activated, anions, such as Cl, present in body fluid 255 and/or products of the reduced metal chloride cathode migrate under the influence of the electric field through the anionic exchange membrane 250 towards the anode 240 in the driving chamber 225.

As with the embodiment disclosed in connection with FIG. 1, fluid delivery device 200 of FIG. 2 may also provide a method of minimizing or preventing the exposure of Zn²⁺ to a patient's body without eliminating the establishment of osmosis in the device. As described above, the steady buildup of ion concentration in the driving chamber 225 induces further water transport via an osmotic effect.

With conventional systems using zinc or similar metals as an anode the ion concentration difference on either side of the ion exchange membrane 250 creates a back-diffusion driving force for ZnCl₂ transport from the driving chamber 225, towards cathode 230. The back diffusion of zinc or a similar metal into body fluid 255 may create the previously discussed responses by the body, such as a toxicological response from the surrounding tissue, influence of the encapsulation behavior, and/or facilitate unwanted protein interaction(s).

Consequently, the anode may be a biocompatible anode, such as the exemplary anodes described in connection with FIG. 1. For example, the anode may be an enzymatic anode that is configured to oxidize glucose. In FIG. 2, the driving chamber 225 may include an aqueous solution of glucose with a sufficient concentration to sustain the anodic current density throughout the operational lifetime of the device 200. Alternatively, the glucose or lactate present in the body fluid may diffuse through the anion ion-exchange membrane and is reduced at the cathode.

The cathode 230, which may be exposed to body fluid 255, may comprise those cathodes discussed in conjunction with the embodiments described in FIG. 1. For instance, cathode 230 may be a metal chloride, such as silver chloride. Alternatively, cathode 230 may be an oxygen reducing cathode. Furthermore, the oxygen reducing cathode 230 may be an enzymatic cathode, which may reduce the ratio of the electro-osmotic engine volume to volume of fluid to be dispensed, compared to conventional systems and system employing larger electrodes, such as a metal chloride.

Although several particular compositions and materials have been disclosed herein, it should be understood that numerous variations thereof are possible as well. For example, each of the fluid reservoirs, bags, bellows, etc., disclosed and described herein can be considered means for housing a beneficial agent. Likewise, each of the pistons, plungers, diaphragms, bladders and bellows, can be considered means for driving the beneficial agent from the housing means. Furthermore, the electrochemical devices, pumps and engines disclosed herein are examples of means for applying pressure to the driving means.

Without further elaboration, it is believed that one skilled in the art can use the preceding description to utilize the present disclosure to its fullest extent. The examples and embodiments disclosed herein are to be construed as merely illustrative and not a limitation of the scope of the present disclosure in any way. It will be apparent to those having skill in the art that changes may be made to the details of the above-described herein. In other words, various modifications and improvements of the embodiments specifically disclosed in the description above are within the scope of the appended claims. Note that elements recited in means-plus-function format are intended to be construed in accordance with 35 U.S.C. § 112 ¶ 6. The scope of the invention is therefore defined by the following claims.

1. A fluid delivery device, comprising:
a fluid reservoir configured to contain a fluid to be dispensed; and
an electrochemical pump configured to facilitate dispensing the fluid from the fluid reservoir, the electrochemical pump, comprising:
a driving chamber;
a first electrode comprising a cathode; and
a second electrode comprising an anode configured to oxidize a fuel present in a body of an animal;
wherein one of the electrodes is disposed within the driving chamber and one of the electrodes is disposed outside of the driving chamber.

2. The fluid delivery device of claim 1, wherein the electrochemical pump is an electro-osmotic pump and the driving chamber is capable of retaining water transported across an ion exchange membrane into the driving chamber to apply pressure to the fluid reservoir.

3. The fluid delivery device of claim 2, wherein the driving chamber displaces a displaceable member upon transportation of water across the ion exchange membrane, such that the displaceable member applies pressure to the fluid reservoir to dispense the fluid.

4. The fluid device of claim 2, wherein the ion exchange membrane is an anionic exchange membrane and the anode is disposed within the driving chamber.

5. The fluid delivery device of claim 2, wherein the ion exchange membrane is a cationic exchange membrane and the cathode is disposed within the driving chamber.

6. The fluid delivery device of claim 1, wherein the fluid comprises a beneficial agent.

7. The fluid device of claim 1, wherein the anode is an enzymatic anode.

8. The fluid delivery device of claim 7, wherein the enzymatic anode is biocompatible.

9. The fluid delivery device of claim 8, wherein the enzymatic anode uses direct electron transfer as an electron transfer mechanism such that the enzymatic anode is chosen from laccase, lactate dehydrogenase, peroxidase, and hydrogenase.

10. The fluid delivery device of claim 8, wherein the enzymatic anode uses mediator electron transfer as an electron transfer mechanism, such that the enzymatic anode is chosen from bilirubin oxidase and glucose oxidase.

11. The fluid delivery device of claim 8, wherein the enzymatic anode is configured to oxidize glucose.

12. The fluid delivery device of claim 8, wherein the enzymatic anode is configured to oxidize lactate.

13. The fluid delivery device of claim 1, wherein the cathode is chosen from a reducible metal chloride cathode and a metal oxide cathode.

14. The fluid delivery device of claim 1, wherein the cathode comprises an oxygen-reducing cathode.

15. The fluid delivery device of claim 14, wherein the cathode is chosen from silver, platinum and metal oxides deposited on a carbon substrate.

16. The fluid delivery device of claim 14, wherein the cathode is an enzymatic cathode which is chosen from bilirubin oxidase, laccase and cytochrome c oxidase.

17. The fluid delivery device of claim 14, wherein the cathode is porphyrin based.

18. The fluid delivery device of claim 1, further comprising a resistor coupled between the first electrode and the second electrode.

19. The fluid delivery device of claim 1, wherein the animal is a human.

20. An implantable device for dispensing a beneficial agent, comprising:
a fluid reservoir configured to contain the beneficial agent; and
an electrochemical pump configured to facilitate dispensing the beneficial agent from the fluid reservoir, the electrochemical pump comprising:
a first electrode comprising a metal chloride cathode; and
a second electrode comprising a glucose oxidation anode.

21. The device of claim 20, wherein the metal chloride cathode is a silver chloride cathode.

22. The device of claim 20, wherein the glucose oxidation anode is configured to be directly exposed to body fluid while the metal chloride cathode is disposed within a chamber separated from the glucose oxidation anode by a cationic exchange membrane.

23. The device of claim 20, wherein the metal chloride cathode is configured to be directly exposed to body fluid while the glucose oxidation anode is disposed within a chamber separated from the metal chloride cathode by an anionic exchange membrane.

24. The device of claim 20, wherein the electrochemical pump is an electro-osmotic pump capable of transporting water across an ion exchange membrane into a first driving chamber to apply pressure to the fluid reservoir.

25. A fluid delivery device, comprising:
means for housing a beneficial agent within the delivery device;
means for driving the beneficial agent from the housing means; and
means for applying pressure to the driving means, wherein the means for applying pressure comprises a first electrode comprising a cathode and a second electrode comprising and enzymatic anode, such that one of the electrodes is directly exposed to body fluid and one of the electrodes is separated from direct exposure to body fluid via an ion exchange membrane.

26. The fluid delivery device of claim 25, wherein the enzymatic anode is biocompatible and is configured to oxidize glucose.

27. The fluid delivery device of claim 26, wherein the cathode comprises a metal chloride.
